(19) United States
(12) Patent Application Publication
(10) Pub. No.: US 2007/0162142 A1
(43) Pub. Date: Jul. 12, 2007

(54) Knee surgery method and apparatus

(75) Inventor: A. Curtis Stone, Aspinwall, PA (US)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(73) Assignee: Vitruvian Orthopaedics, LLC, Aspinwall, PA (US)

(21) Appl. No.: 11/454,135

(22) Filed: Jun. 15, 2006

Related U.S. Application Data
(60) Provisional application No. 60/690,705, filed on Jun. 15, 2005.

(51) Int. Cl. A61F 2/38

(52) U.S. Cl. 623/20.14

(57) ABSTRACT

A device for positively clamping to the patella for use in knee surgical procedures such as total or partial knee arthroplasty to aid in determining the position of the center of the patellar implant and the amount of resection of the posterior patella for to achieve optimal tracking of the patello-femoral joint with the least amount of soft tissue releases. In one embodiment, the device includes a docking mechanism for attaching a posterior patella guide and one or more navigation devices, sensors and/or fiduciaries to monitor the position of the patella in the full range of motion and to aid in selecting the optimal position of the patella and patellar implant.

### CROSS-REFERENCE TO RELATED APPLICATIONS

This application claims the benefit of U.S. Provisional Patent Application 60/690,705, filed Jun. 15, 2005, and entitled “METHOD AND DEVICE FOR AFFIXING NAVIGATIONAL AND SURGICAL DEVICES TO THE PATELLA DURING KNEE SURGERY,” which is incorporated herein by reference in its entirety.

### TECHNICAL FIELD

The present invention relates to systems and methods for orienting and/or positioning medical instruments and/or implants and/or cutting bone, and more particularly, for orienting and/or positioning medical instruments that are used to prepare a surgical site via alignment or resection for positioning and/or correct implant placement and for instruments that are used for delivering medical implants to a prepared site.

### BACKGROUND

The knee is a complicated, marvelous structure, made up of a combination of bones, muscles, tendons, ligaments and other soft tissue. Generally, the human knee joint comprises three bones, the femur, the tibia and the patella, which each have a smooth surface for articulation on an adjacent surface of at least one other bone. At its distal extremity, the femur has an articulation surface with medial and lateral convex condyles separated posteriorly by an intercondylar groove running generally in the anterior-posterior direction. The condyles join at the distal-anterior face of the femur to form a patellar surface having a shallow vertical groove as an extension of the intercondylar groove.

The patella includes on its posterior face an articulation surface having a vertical ridge separating medial and lateral convex facets. These facets articulate against the patellar surface of the femur and against the medial and lateral condyles during flexing of the knee joint. The vertical ridge rides within the intercondylar groove to prevent lateral displacement of the patella during flexing of the knee joint. The tracking of the patella can be influenced by different things. Most of it is influenced by the anatomical shape of the knee. The following can all contribute to the maltracking of the patella: shallowness of the femoral groove, the angle of the knees (knock-knees), rotation of the hips, foot positioning on the floor (pronation or flat feet), weakness of the quadriceps, the shape of the patella and malalignment of implants during knee reconstruction.

At its proximal end, the tibia includes an articulation surface having medial and lateral meniscal condyles that articulate against the medial and lateral condyles, respectively, of the femur. The mutually engaging articulation surfaces of the femur and the patella together form the patello-femoral joint and the mutually engaging articulation surfaces of the femur and tibia form the tibiofemoral joint. These two functional joints form the anatomical knee joint.

Because of disease or trauma, all or part of one or more of the articulation surfaces of the knee joint may fail to perform properly. This can lead to a need or desirability for replacement of the defective natural articulation surface with a prosthetic articulation surface of an implantable prosthesis. A range of orthopedic implants is available, including patella prostheses used to replace the natural articulation surface of the patella. Such replacements may be accomplished by surgically resecting the patella to remove the posterior portion of the bone, leaving a planar bony surface to which a patellar prosthesis is affixed. The patellar prosthesis typically has an affixation surface affixed to the resected bony surface of the patella with, for example, bone cement. The prosthesis also typically includes an articulation surface that may be made of a biocompatible synthetic polymer material, such as ultrahigh molecular weight polyethylene. The patella includes on its posterior face an articulation surface having a vertical ridge separating medial and lateral convex facets. These facets articulate against the patellar surface of the femur and against the medial and lateral condyles during flexing of the knee joint. The vertical ridge rides within the intercondylar groove to prevent lateral displacement of the patella during flexing of the knee joint.

Total and partial knee replacement surgery has been performed for over thirty years, providing relief for those with chronic, debilitating pain caused by degeneration of the cartilage in the knee, angular deformities, or serious knee injuries. The surgery requires the removal of the pathologic joint articular surface and subsequent replacement with knee prostheses. A small portion of bone in the knee is removed, and covered with a combination of metal and plastic components to form the new surfaces of the joint. With the new surfaces sliding together smoothly, the bone no longer rubs together and pain is relieved. Today, this procedure is very popular and well recognized for producing excellent clinical results for patients. In the United States, there are approximately 200,000 to 300,000 patients undergoing total knee replacement annually.

In total knee arthroplasty (TKA) the bone and cartilage at the distal end of the femur and at the proximal end of the tibia are removed and replaced with plastic, metal, and/or ceramic parts. Frequently, the articulating surfaces of the patella and sulcus (opposing inner surface) may also be replaced. Because of the tendons connected to the patella, it is generally advisable to replace only the articulating surface. A less drastic procedure, partial knee arthroplasty (PKA), can be performed where the damage to the cartilage is restricted to one confined area of the knee. In the event that the osteoarthritis is confined strictly to the patello-femoral joint, a PKA is performed whereby the underside of the patella may be replaced with a patellar implant and the counter surface receives a metal trochlear groove implant. In case of isolated anterior knee pain wherein the native sulcus is not diseased or damaged, it may be useful to replace only the patella's cartilage with a patella implant.

Increasing numbers of total knee replacements have resulted in the demand to improve surgical techniques. One of the long-term outcomes of this procedure is the mechanical wear of the prosthetic components; therefore, this limits the longevity of the knee replacement and a subsequent revision may be required. The revision may be more complicated and more costly than the initial surgery.

Factors affecting prosthetic lifespan vary. One of the most important factors is the accuracy of prosthesis placement and the restoration of surrounding muscle and ligament balance. This relies mostly on the orthopedic surgeon's skill and judgment. However, research has shown that incorrect placement still occurs even with the most skilled and experienced surgeons. The chance for incorrect prosthetic placement will be greater for a less skilled or inexperienced surgeon.

Correct positioning of surgical instruments and implants, used in a medical and/or surgical procedure, with respect to the patient's anatomy, is an important factor in achieving the goal of the procedure. For example, in certain medical implant procedures such as total hip arthroplasty and TKA, the accurate preparation of the surgical site and the optimal orientation of the surgical implant enhance initial function and the long term survivability of that implant.

The outcome of TKA surgery is particularly sensitive to variations in surgical technique. Incorrect orientation of implants and improper alignment can lead to accelerated implant wear, loosening, and sub-optimal functional performance. Even the most elaborate mechanical instrumentation systems rely on visual cues to confirm the accuracy of the limb and implant alignment, particularly rotational orientation.

There currently are mechanical alignment guides provided by the implant system manufacturers and commercially available computer-aided systems (CAS) and image-guided surgical systems (CIGS) that provide information and aid the correct positioning of preparation instruments and surgical implants.

Proper anatomic function of the knee joint is facilitated by proper placement of the prosthetic articulation surface or surfaces during surgery. If the affixation surface is properly affixed to the bony surface of the patella, the prosthetic articulation surface of the patella tracks the intercondylar groove and anterior patellar surface of the femoral prosthesis. During surgery, it is also important and sometimes difficult to mount the patellar prosthesis with proper angular placement.

Knee arthroplasty procedures require the resection of the natural articulation surface of the patella for replacement thereof with a prosthetic patella component. Subsequently, through known procedures, the resected surface of the patella is reamed to form a cavity that can accept the patella prosthesis.

The most common patellar implant is usually fashioned from a hard plastic (ultra high molecular weight polyethylene) in the form of a button, with or without a metal base element. This implant replaces the anterior side of the patella, adjacent the femoral condyles. To implant such a prosthesis, the posterior surface of the patella is resected to produce a flat surface on which the prosthesis is mounted. The patellar implant is configured to slide up and down either the natural trochlear groove (when it was not replaced in a PKA) or an artificial trochlear groove of the femoral component, which is metal or ceramic.

Many methods have been used in the prior art for attaching a patella implant to the native patella underside. A typical patellar implant includes pegs (usually three) on its rear surface, which must be perfectly aligned before attachment. This is a difficult task requiring surgical precision to ensure a perfect alignment so that the patella implant tracks up and down against the counter surface groove without diverting tangentially. Improper tracking may occur for example when the implant is in the correct location on patella but the pegs are not (e.g., instead of being positioned at “noon-four o'clock-eight o'clock”, the pegs are at “one o'clock-five o'clock-nine o'clock), thus causing a grooved patella implant to misalign. This patellar misalignment and resulting abnormal tracking may produce significant shearing forces and excessive contact stress that may cause degeneration of the natural articular cartilage or of the patellar implant when articulating against the metal groove in case of replacement of the natural groove. Additionally or alternately, the pegs may be aligned with the correct orientation but they are shifted slightly to the right or left so that the implant's dome area is not aligned with the target recess of the artificial or natural groove.

It would be desirable to have patellar anatomic position locator and resection instrument designs that reduced or eliminated problems associated with medial-lateral or angular misplacement of the patellar prosthesis.

A number of clamping devices are known to exist. For example, U.S. Pat. Nos. 5,284,482; 5,129,908; and 4,706,660 all disclose various patella clamping devices. These patents describe clamp devices that require two hand operation: one hand closes the clamp while the other uses a thumb screw or a similar device to lock the clamp in the closed position. The design of the disclosed clamps is such that the closing motion of the clamp is non-linear.

Patella clamping devices generally include two jaw elements that close together to engage and hold firmly the prosthetic patella component and the natural patella. It is desirable for the jaws to close together in a consistent manner with a linear application of force so that the components are not drawn out of alignment while the load is applied. Once the jaws are closed to apply the load to the two components, the jaws must be held together firmly with a constant application of pressure. To accomplish this, it is often necessary to lock the jaws in place.

Many patella clamps have locking devices to maintain the jaws of the clamp in the closed, load applying position. Known clamp designs, such as those described in the references noted above, usually require the surgeon to maintain pressure on the clamping jaws with one hand while using the other hand to actuate the locking mechanism. The necessity for the surgeon to use two hands during the locking procedure can be inconvenient. It would thus be advantageous to provide a clamping device that enables the linear application of load and which enables locking of the device using only one hand.

Many patella clamps also do not permit the clamping members to close with a vertical or linear application of load. A non-linear closing motion of a clamp could result in the application of forces which cause misalignment of the patella components while the clamping load is applied.

Computer technology has been introduced to assist in more accurate positioning of prosthetic components, allowing the surgeons to make informed decisions intraoperatively for precise prosthetic placement. This allows physiologic muscle and ligament balance which is an important factor in prolonging the prosthetic lifespan and reducing the chances for an early revision arthroplasty.

Surgeons can use this real-time data to correct the procedure intraoperatively. This is much different from non-CAS surgery as the prosthetic evaluation by X-ray is done post-operatively. Should there be any incorrect prosthetic alignment, there is a greater likelihood of requiring a subsequent revision. There is a greater risk for post-operative complication as well as increased costs. In this case of incorrect prosthetic alignment from non-CAS surgery, the patient and physician must accept the surgical outcome, including the likelihood of shortened total knee arthroplasty lifespan.

There has been much emphasis placed on the utilization of advanced computer and imaging technologies to aid in the correct anatomic placement of implants during reconstruction and resurfacing procedures, such as total hip and total knee reconstruction. In the total knee reconstruction procedure the navigation devices are affixed to the femur, the tibia and the instrumentation to assure correct and congruent alignment with the anatomic axis' and with each component. However, to date there has been no substantial solution to include the patella within the technology other than for the surgeon to lateralize the component placement and/or conduct extensive soft tissue releases. There has not been a substantive means, other than surgeon visual and physical evaluation, to measure the position of the patella in reference to the patello-femoral groove. There is a need to be able to utilize the advanced systems to quantify the position of the patella in the static positions during maximum flexion and extension and the tracking throughout the full range of motion.

Accordingly, there is a need in the art for a patella alignment and clamping device that improves the placement and resection of the patella to avoid complications associated with malalignment. Additionally, there is a need in the art for a patella clamp that can be used with positioning aides for greater accuracy. Additionally, there is a need in the art for a clamping cutting guide device/instrument to operatively couple a device, transmitter, and/or fiduciaries to monitor the position of the patella in the full range of motion, help determine the position of the center of the patella implant and the amount of resection of the posterior patella for optimal tracking of patello-femoral joint with the least amount of soft tissue releases.

### SUMMARY

The present invention, according to one embodiment, is an apparatus for positively clamping to a patella for placement of a patellar insert during a knee surgery procedure. The apparatus includes first and second handles each having a proximal portion and a distal portion. The handles are pivotally connected together in a scissors-like arrangement. The apparatus further includes opposed first and second clamping members coupled to the first and second handle member distal portions, respectively. Because of the pivotal connection between the handle members, movement of the first handle member proximal portion toward the second handle member proximal portion results in movement of the first clamping member toward the second clamping member. The apparatus also includes a docking component coupled to either the first or second clamping members, or to both. The docking component is adapted for movement relative to the clamping members and can receive one or more surgical navigation devices. A guide member is coupled to the docking component and can extend from an anterior to a posterior side of the patella.

The present invention, in another embodiment, is a method of using a patella guidance clamp device in a patella alignment and resection phase of a total or partial knee arthroplasty procedure. The patella guidance clamp includes a docking mechanism adapted to which one or more surgical navigation aids or tracking devices and a posterior patella alignment guide can be attached. The method includes applying the and locking the clamp to a surgical patient's patella, coupling the docking mechanism to the clamp, coupling one or more navigation aids or tracking devices to the docking mechanism, and coupling, positioning and locking the posterior patella alignment guide to define a patella midline based on a pre-determined patella implant position. Then, using the attached navigation aid(s) and/or tracking device(s), a predetermined patellar implant position can be validated. If this predetermined position is found to be not optimal, the patellar implant position is adjusted. The depth of resection or reaming of the patella for optimal patella implant placement is then determined.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is an anterior partial schematic view of an exemplary patella guidance clamp, according to one embodiment of the present invention, shown clamped onto a patella during a knee surgery.

FIG. 2 is a schematic view of a handle portion of an exemplary patella guidance clamp according to one embodiment of the present invention.

FIGS. 3A and 3B are schematic distal views of a clamping portion of exemplary embodiments of the patella guidance clamp of the present invention, depicted positioned on and clamped to a patient's patella and including exemplary surgical navigation aid devices attached thereto.

FIG. 4 is a partial schematic anterior view of a clamping portion of an exemplary patella guidance clamp according to one embodiment of the present invention, shown positioned on a patella.

FIG. 5 is a partial schematic posterior view of a clamping portion of an exemplary patella guidance clamp according to one embodiment of the present invention, shown positioned on a patella.

FIG. 6 is a perspective view of an exemplary surgical orientation device of the type which may be used with the patella guidance clamp according to one embodiment of the present invention.

FIGS. 7A-C is a flowchart describing the use of the patella guidance clamp of FIGS. 1-5 in conjunction with a surgical navigation aid.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

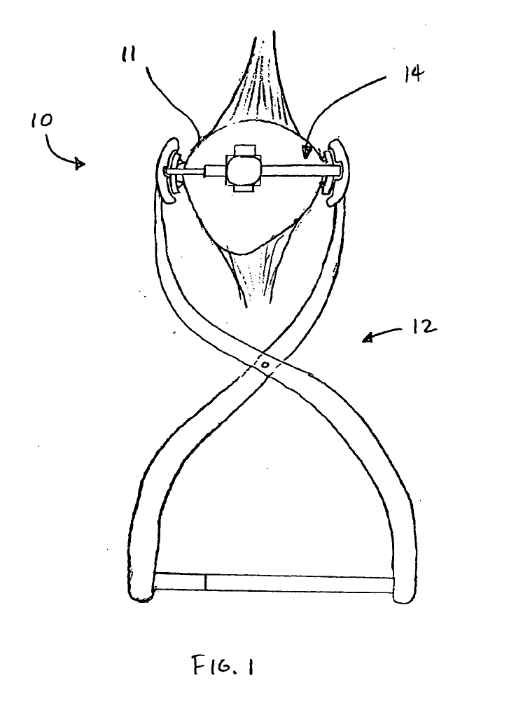

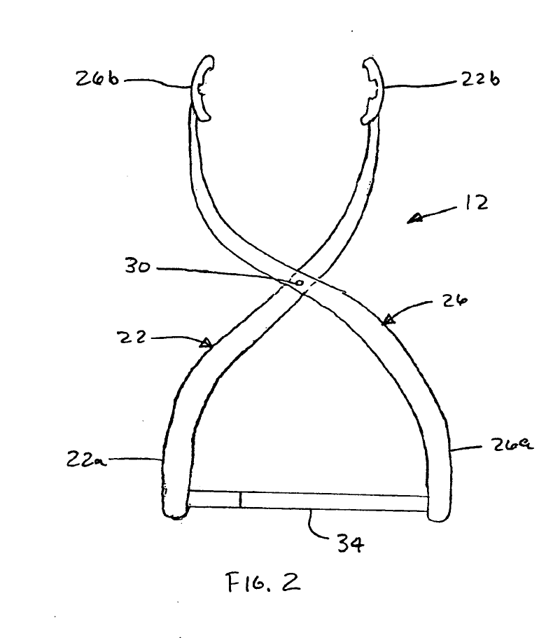

FIG. 1 an anterior partial schematic view of an exemplary patella guidance clamp 10 according to one embodiment of the present invention, positioned and clamped onto a patella 11 as during a knee surgery procedure such as a TKA. As shown in FIG. 1, the patella guidance clamp 10 includes a handle portion 12 and a clamping portion 14.

The handle portion 12 is adapted to mate with and maintain a load on the clamping portion 14, which is, in turn, adapted to engage and exert a clamping force on the natural patella 11 and/or a patella prosthesis during the knee arthroplasty procedures. In one embodiment, the clamping portion 14 may be permanently attached to the handle portion 12. Alternatively, the clamping portion 14 may be detachable from the handle portion 12. In one embodiment, the clamp 10 does not include the handle portion 12. In this embodiment, any of a variety of instruments known in the art may be used to attach the clamping portion 14 to the patella 11.

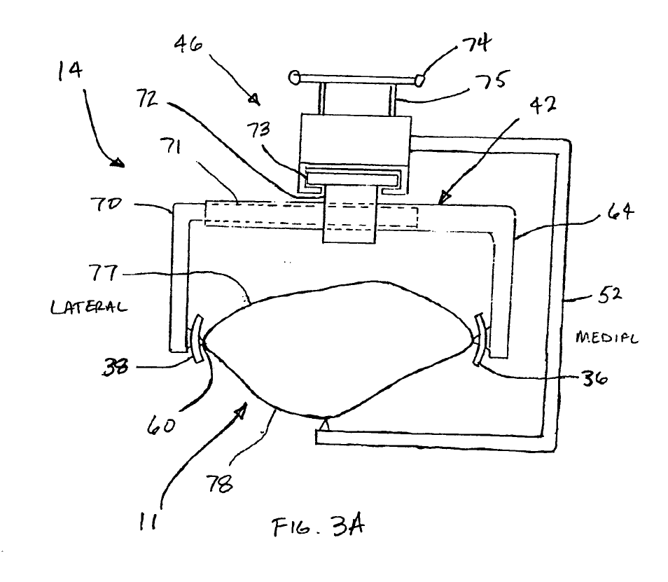

FIG. 2 depicts, schematically, the handle portion 12 of the patella guidance clamp 10 depicted in FIG. 1. As can be seen in FIGS. 1 & 2, the handle portion 12 includes a first handle member 22, a second handle member 26 joined to the first handle member 22 at a pivot point 30 to form a scissors-like arrangement, and an optional selectively engageable mechanism 34 (e.g., ratchet, screw or other locking device). The first and second handle members 22 and 26 each has a proximal portion 22a and 26a, respectively, and a distal portion 22b and 26b, respectively.

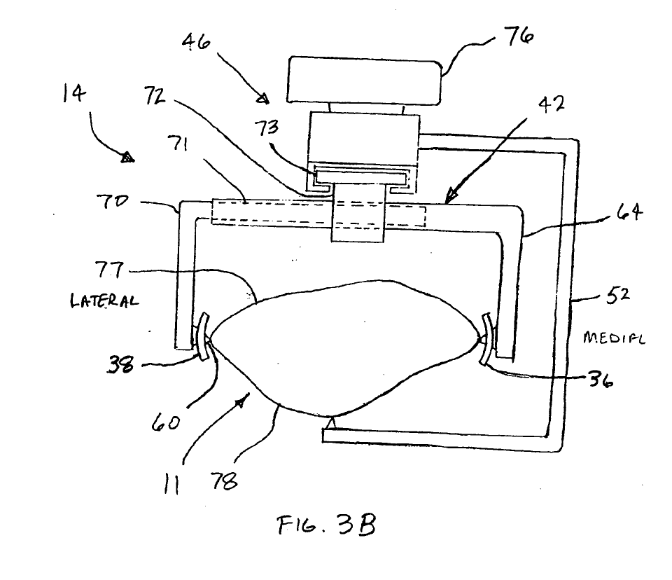

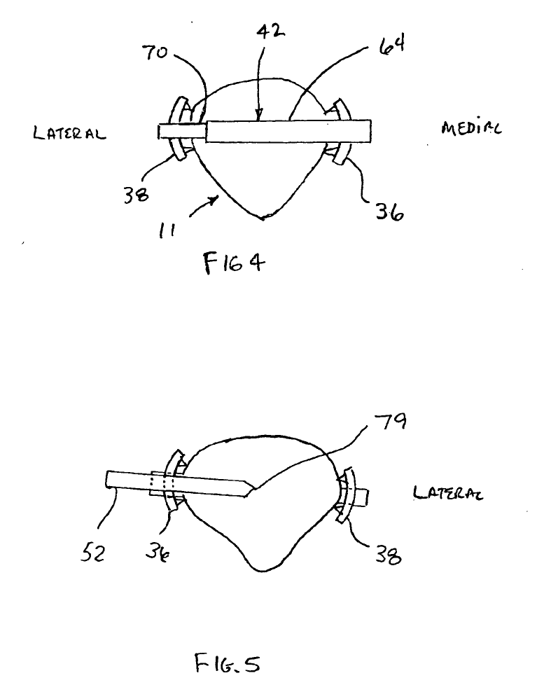

FIGS. 3A and 3B schematically show, from a distal view, the clamping portion 14 positioned onto and clamped to the patella 11. As can be seen in FIGS. 3A and 3B, the clamping portion 14 includes opposed first and second jaw members 36 and 38, a linear bearing 42, a docking mechanism 46, and a posterior guide 52. Alternatively, in other embodiments, the linear bearing 42 may be omitted.

The first and second handle distal portions 22b and 26b are adapted to mate with and engage the first and second jaw members 36 and 38, respectively. Due to the scissors-like orientation of the handle members, a force applied to close together the first and second handle proximal portions 22a and 26a results in closing of the handle distal portions 22b and 26b. This forces the first and second jaw members 36 and 38 toward one another which, in turn, creates a clamping force when the clamping portion 14 is positioned on the patella 11 as in FIG. 1. In one embodiment, the jaw members 36 and 38 may include one or more teeth 60 for engaging and gripping the patella 11. In one embodiment, the selectively engageable mechanism 34, when engaged, permits closure, but prevents further opening, of the handle members 22 and 26.

In one embodiment of the present invention, the linear bearing 42 includes a first elongated member 64 that is rigidly attached to the first jaw member 36, and a second elongated member 70 rigidly attached to the second jaw member 38. The first elongated member 64 is sized and shaped (e.g., as a rectangular tube, channel, etc.) to receive the second elongated member 70, such that a distal portion 71 of the second elongated member 70 may travel within the first elongated member 64. In another embodiment, the linear bearing 42 may be attached to the first and second handle members 22 and 26 at a location intermediate of the pivot point 30 and the first and second handle member distal portions 22b and 26b. The linear bearing 42 operates to ensure application of the clamping load by the clamping portion 14, while at the same time beneficially allows the first and second jaw members 36 and 38 to close in a coaxial motion.

In one embodiment, a biasing element (e.g., spring) (not shown) is disposed within the linear bearing 42 and operates to bias the first and second jaw members 36 and 38 in an open position. In other embodiments, this biasing element is located elsewhere on the patella guidance clamp 10, or is omitted.

In one embodiment, proper coaxial closing action of the clamping elements can be achieved by forming a joint (not shown) in the second elongated member 70 that travels within the first elongated member 64 of the linear bearing 42, thus forming segmented portions of this elongated member. The segmented portions of the jaw member are thus able to articulate within the bearing member.

In one embodiment, the clamping portion 14 includes a lock (e.g., a set screw) (not shown) for fixing the respective positions of the first and second elongated members 64 and 70.

The patella guidance clamp 10 of the present invention can be advantageously attached in such an unobtrusive manner as to enable access of the specific implant systems patella resection clamp or can be permanently or temporarily operatively coupled with the patella resection clamp.

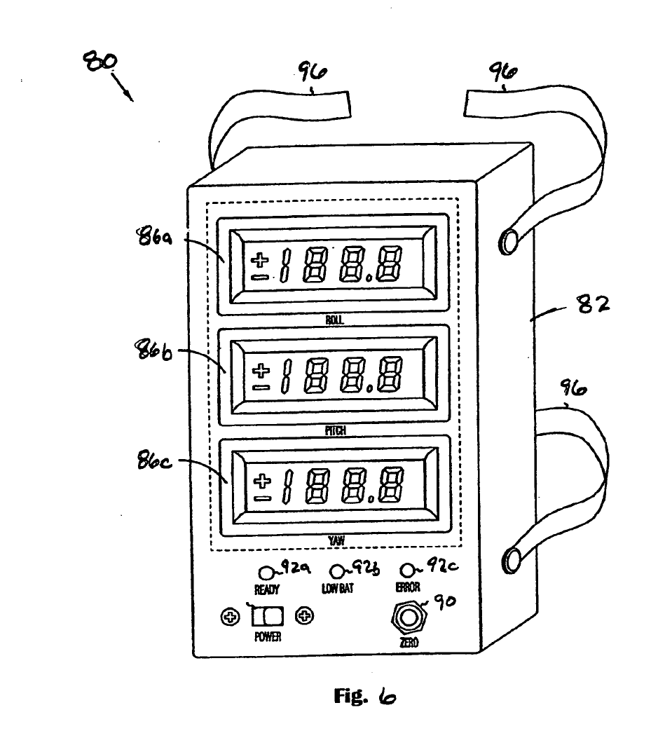

As shown in FIGS. 3A and 3B, in one embodiment, the docking mechanism 46 is operatively coupled to the linear bearing 42. Alternatively, the docking mechanism 46 may, in other embodiments, be operatively coupled to another component of the clamping portion 14. The docking mechanism 46 may be fixed to the clamping portion 14, or, alternatively, may be removable. FIG. 4 is a partial schematic anterior view of another embodiment of the clamping portion 14 shown positioned on the patella, with the docking mechanism 46 omitted.

The docking mechanism 46 is adapted to be mobilized in the medial/lateral and superior/inferior directions while coupled to the linear bearing 42 or other element of the clamping portion 14 and to be locked into place (e.g., via a set screw) once a desired position is attained. As shown in FIGS. 3A and 3B, in one embodiment, the docking mechanism 46 may be operatively coupled to a first slide member 72 that permits medial and lateral movement along the linear bearing 42, and a second slide member 73 oriented perpendicular to the linear bearing 42 and which permits superior and inferior translation of the docking mechanism 46. It is important to note, however, that the docking mechanism 46 is not limited to the structure depicted in FIGS. 3A and 3B. To the contrary, any structure that permits both medial/lateral and superior/inferior translation of the docking mechanism 46 relative to the linear bearing 42 may be employed.

The docking mechanism 46 is further adapted for the attachment of navigation or other surgical devices such as, for example, tracking sensors, fiduciaries, or an electronic orientation guide. FIG. 3A shows an exemplary embodiment in which fiduciaries 74 are attached to the docking mechanism 46. In one embodiment, navigation system fiduciaries can be attached to parallel pins 75 protruding perpendicular to the docking mechanism's plane of translation. These pins 75 may also be sized and configured to utilize existing attachment schemes of the navigation systems, and may be permanently attached to the docking mechanism 46 or may be removable (e.g., with a threaded or locking screw attachment). According to other embodiments, the docking mechanism 46 may include one or more other attachment components sized and shaped to mate with attachment schemes of different electronic navigation/orientation guides or surgical instruments. FIG. 3B shows an exemplary embodiment in which an electronic navigation device 76 is affixed to the docking mechanism 46 in the medial/lateral and superior/inferior planes.

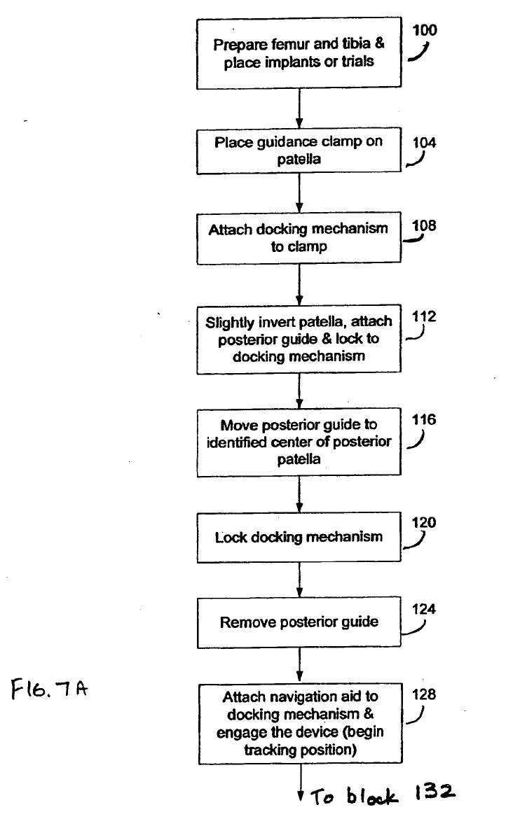

The posterior guide 52 is, in one embodiment, operatively coupled to but removable from the docking mechanism 46. The posterior guide 52 is adapted to be mobilized medially and laterally, as well as in the superior/inferior directions, while operatively coupled to the docking mechanism 46 or other component of the clamping portion 14, and to be locked into place once a desired position is attained. Any locking means (e.g., set screw) that prevents rotation and facilitates reproducible attachment placement may be used to lock the posterior guide 52 to the docking mechanism 46. As shown in FIGS. 3A & B, in one embodiment, the posterior guide 52 is sized and shaped to extend from an anterior side 77 of the patella to a posterior side 78 of the patella 11. The posterior guide 52 operates to enable the identification of a midline or other chosen position on the posterior patella 78 as the referenced center of an artificial/replaced patella. FIG. 5 depicts a partial schematic posterior view of a clamping portion of an exemplary patella guidance clamp according to one embodiment of the present invention, showing the posterior guide 52 identifying a chosen position 79 on the posterior patella 78.

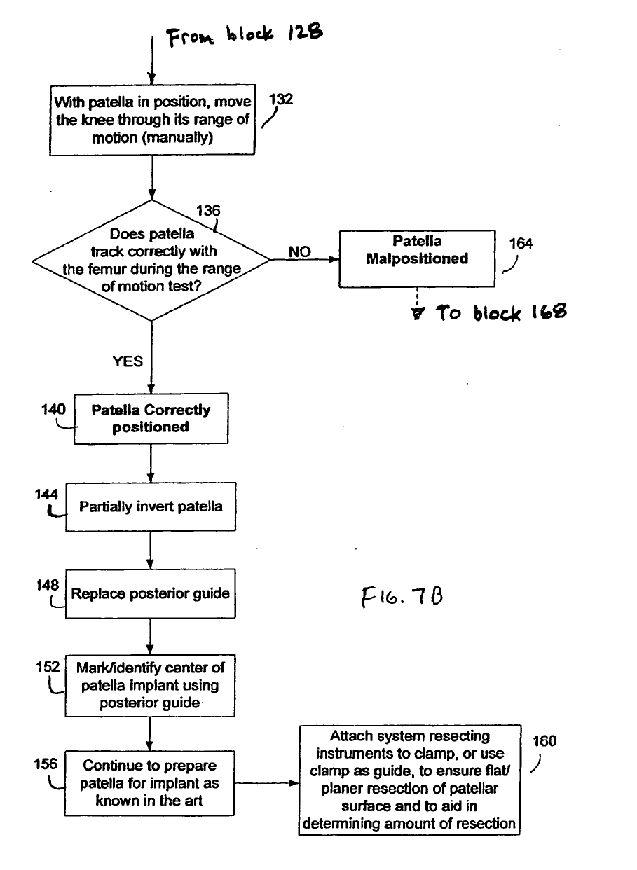

FIG. 6 shows an exemplary electronic surgical orientation guide device 80 of the type which may be used with the patella guidance clamp of the present invention. The surgical orientation guide depicted in FIG. 6 is described in detail in U.S. Publication 2005/0021044 A1, which is hereby incorporated by reference in its entirety. As described therein and as shown in FIG. 6, the device 80 includes a housing 82, displays 86, a zero button 90, and indicator lights 92. The housing 82 contains electronic circuitry and components necessary for operation. The displays 86, in the embodiment shown in FIG. 6, include a ROLL display 86a, a PITCH display 86b, and a YAW display 86c. These displays 86 provide an indication of the angular orientation of the device in three dimensions. The device 80 may also include displays showing linear (i.e., lateral, medial, proximal and/or distal) movement of the device 80. In one embodiment, the device includes the sensors for providing position and orientation signals. Indicator lights (e.g., LEDs) may be integrated into the housing 82 to indicate when a positional property of interest, such as a angle, has been reached and/or not reached and/or exceeded. The surgical orientation guide device 80 of FIG. 6 may further include means (e.g., attachment straps 96 or clips) connected to the housing 82 and configured to allow attachment of the device 80 to the docking mechanism 46 of the patella guidance clamp 10 of the present invention.

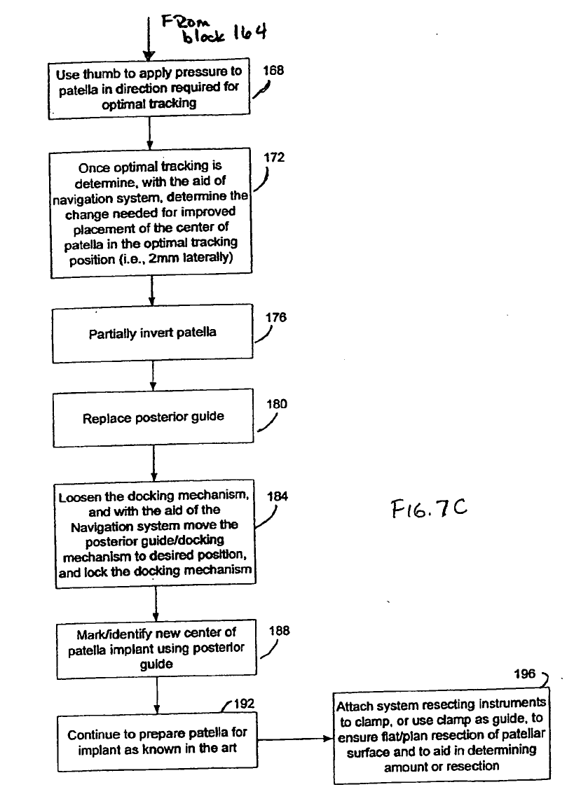

FIGS. 7A-C show a flowchart describing an exemplary method of use of the patella guidance clamp in conjunction with a surgical navigation aid device, such as, but not limited to, the device 80 shown in FIG. 6. As shown in FIGS. 7A-C, after the femur and tibia have been prepared and the implants or trials have been placed according to procedures known in the art (see block 100), the guidance clamp 10 is placed onto and clamped to the patella 11 (block 104), the docking mechanism 46 is attached to the clamp 10 (block 108), and the posterior guide 52 is attached and locked to the docking mechanism 46 (block 112). The posterior guide 52 is then moved to a previously identified center of the patella 11 (block 116) and the docking mechanism 46 is locked into place (block 120). The posterior alignment guide 52 may then be removed (block 124).

The surgical navigation device is then attached to the docking mechanism 46 and, with the knee in extension, the navigation device is set or registered (i.e., zeroed) (block 128). The knee is then flexed throughout the full range of motion (block 132) and the tracking of the patella 11 within the patello-femoral groove can be evaluated using the navigation device (block 136). The navigation systems can track and measure the movement of the patella 11 throughout the full range of motion of the knee.

For example, the surgeon may take particular notice of the anterior plane and the medial/lateral angular displacement of the patella 11 as indicated by the navigation device or system. As is known in the art, excessive displacement (i.e., displacement exceeding well-known parameters) is an indication of non-optimal patella tracking. If the tracking is deemed optimal, the posterior guide 52 is reattached to the docking mechanism 46 (blocks 140-148), the position marked (by drill, marking pen, bovie, etc. as know in the art) (block 152) and the resection procedure continues as proscribed in the art (blocks 156-160).

If, in this example, the tracking of the patella is not optimal, positive pressure may be applied to the patella during the full range of motion and the optimal linear and angular position can be determined (block 168). The adjustment necessary to achieve the optimal position of the patella (e.g., 2 mm laterally) can then be determined with the aid of the navigation device or system (block 172). For example, in one exemplary embodiment, the navigation device may be a special-purpose device capable of measuring and displaying roll and medial/lateral linear displacement, and may be oriented along or parallel to the superior-inferior axis. If, in this example, the patella 11 experiences medial lift during the range of motion, the navigation device will be able to detect and display this medial displacement by indicating a change in its roll display. If the surgeon deems the movement to be excessive (based on parameters known in the art), the surgeon may apply lateral pressure to the patella 11, usually with the thumb, to displace the patella laterally. The surgeon then may again move the patient's knee through the full range of motion and monitor patellar displacement using the navigation device. This procedure is repeated until the medial lift is reduced to acceptable levels. The navigation system may then automatically record this patellar position resulting in optimal tracking, or the surgeon may make note of the necessary adjustment for optimal position (i.e., the amount he or she had to move the patella 11 from the predetermined position to achieve optimal tracking) using the navigation device, which displays the medial/lateral linear displacement of the device from the pre-determined (i.e., zero) position. The posterior guide 52 may then be replaced (depending upon the tracking system utilized) (blocks 176-180), and the docking mechanism 46 and posterior guide 52 may be moved to the optimal position as identified by the tracking/navigation system (block 184). The new center of the patellar implant and/or center of resection based on this optimal position is then marked using the posterior guide 52 (block 188).

The preparation of the patella for the patellar implant can then continue according to methods known in the art (block 192). In addition, the resecting instruments may be attached to the patella guidance clamp 10 via the docking mechanism 46 and/or the clamp 10 may be used as a guide to ensure a flat or planar resection of the patellar surface or to aid in determining the proper amount of resection (blocks 160, 196).

The clamping and docking mechanisms may be retained on the patella to verify correct position and tracking of the patella 11 once all the components have been implanted. Additionally, the patella guidance clamp 10 and docking mechanism 46 with navigation aids can be advantageously used in conjunction with additional navigation aids coupled to the patient's femur and/or tibia to further assist in obtaining optimal tracking of the patella 11.

This invention can be engaged prior to the posterior resection cut of the patella or after initial or subsequent posterior resection cut of the patella. Various modifications and additions can be made to the exemplary embodiments and methods discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

I claim:

1. An apparatus for clamping to a patella during a knee surgery procedure, comprising:
a first handle member having a proximal portion and a distal portion;
a second handle member having a proximal portion and a distal portion and pivotally coupled to the first handle member;
a first clamping member operatively coupled to the first handle member distal portion;
a second clamping member disposed opposite the first clamping member and operatively coupled to the second handle member distal portion, whereby a pivoting movement of the first handle member proximal portion toward the second handle member proximal portion results in movement of the first clamping member toward the second clamping member;
a docking component operatively coupled to one of the first or second clamping members, the docking component being adapted for movement relative to the clamping members and to receive one or more surgical navigation devices; and
a guide member operatively coupled to the docking component and sized to extend from a patella anterior side to a patella posterior side.

2. A method of performing a knee arthroplasty procedure, the method comprising:
applying a patella guidance clamp to a patella, the patella guidance clamp including a docking mechanism adapted for receiving one or more surgical navigation aids or tracking devices and a posterior patella alignment guide;
locking the patella guidance clamp onto the patella;
coupling the docking mechanism to the patella guidance clamp;
coupling one or more navigation aids or tracking devices to the docking mechanism;
defining a patella midpoint corresponding to a pre-determined patella implant position using the posterior patella alignment guide;
quantifying and validating the pre-determined patella implant position using the one or more navigation aids or tracking devices;
adjusting the patella implant position if the predetermined position is not optimal; and
determining the depth of resection or reaming of the patella for optimal patella implant placement.
